(19) United States
(12) Patent Application Publication
(10) Pub. No.: US 2025/0347688 A1
(43) Pub. Date: Nov. 13, 2025

(54) Glucose Monitoring Device with Integrated Visual and Tactile Alert Mechanisms

(71) Applicant: JoAnne C. Davidson, Amarillo, TX (US)

(72) Inventor: JoAnne C. Davidson, Amarillo, TX (US)

(21) Appl. No.: 18/939,989

(22) Filed: Nov. 7, 2024

Related U.S. Application Data
(60) Provisional application No. 63/643,458, filed on May 7, 2024.

(51) Int. Cl. G01N 33/543; G01N 33/66

(52) U.S. Cl. CPC G01N 33/54386; G01N 33/66

(57) ABSTRACT

The present invention is a glucose monitoring device designed specifically for deaf individuals. The device comprises a housing containing components such as glucose monitoring circuitry for measuring glucose levels in blood samples. The front surface features a display screen that shows glucose readings and other relevant data. The housing is equipped with multiple bright flashing lights and a vibration motor to provide both visual and tactile alerts when glucose levels are abnormal. The alerts persist until acknowledged by pressing an acknowledgement button. Additionally, the device includes a pivotal stand on the rear surface, enabling the device to be angled for optimal visibility of alerts. The system also incorporates a wireless communication channel for transmitting real-time glucose measurements to a remote computing device, facilitating monitoring by users, guardians, or medical practitioners.

### CROSS-REFERENCE TO RELATED APPLICATION

The present application claims priority to, and the benefit of, U.S. Provisional Application No. 63/643,458, which was filed on May 7, 2024 and is incorporated herein by reference in its entirety.

### FIELD OF THE INVENTION

The present invention generally relates to the field of glucose monitoring device. More specifically, the present invention relates to a novel glucose monitor device designed specifically for deaf individuals. The device is equipped with multiple bright flashing lights and a vibration motor to provide both visual and tactile alerts when glucose levels are abnormal. The alerts persist until acknowledged by pressing an acknowledgement button. Additionally, the device includes a pivotal stand on the rear surface, enabling the device to be angled for optimal visibility of alerts. Accordingly, the present disclosure makes specific reference thereto. Nonetheless, it is to be appreciated that aspects of the present invention are also equally applicable to other like applications, devices, and methods of manufacture.

### BACKGROUND

By way of background, glucose monitors are essential devices used by individuals with diabetes to measure and manage their blood glucose levels. Glucose monitors typically alert users when their glucose levels are too high or too low by sounding an audible alarm and briefly illuminating the display screen. However, these conventional glucose monitors do not cater to the specific needs of deaf individuals. For a deaf person, the audible alarm is ineffective as they cannot hear it. Additionally, the brief illumination of the display screen is often too subtle to attract their attention, especially in a medical emergency when immediate awareness and action are critical. Maintaining proper glucose levels is vital for avoiding severe health complications, and timely alerts play a crucial role in this process.

The limitations of current glucose monitors are particularly concerning during sleep. Deaf individuals are unable to hear alarms, and subtle visual cues are not sufficient to wake them or catch their attention. This presents a significant risk, as a failure to promptly address abnormal glucose levels during the night can lead to dangerous health situations. Individuals desire an improved glucose monitoring device that effectively accommodates the needs of deaf individuals.

Therefore, there exists a long felt need in the art for an improved glucose monitoring device designed specifically for deaf individuals. There is also a long felt need in the art for a novel and inclusive glucose monitoring for deaf individuals by combining conventional functionality with enhanced alert mechanisms. Additionally, there is a long felt need in the art for a unique glucose monitoring device that includes bright flashing lights and continuous vibrations to provide effective notifications of abnormal glucose levels. Moreover, there is a long felt need in the art for a unique glucose monitoring system that facilitates monitoring of glucose by users, guardians, or medical practitioners. Finally, there is a long felt need in the art for a uniquely designed glucose monitoring device that enhances safety and promotes independence and confidence in managing diabetes for deaf individuals.

The subject matter disclosed and claimed herein, in one embodiment thereof, comprises a glucose monitoring device for deaf individuals. The glucose monitoring device includes a housing configured to house electronic components for monitoring glucose levels in a blood sample, a display screen on a front surface of the housing for displaying glucose level readings, a plurality of bright flashing lights disposed on the housing, wherein the lights are adapted to flash in bright colors to alert a user when the measured glucose level is abnormal, a vibration motor within the housing adapted to provide a tactile alert when the measured glucose level is abnormal, an acknowledgement button on the housing for deactivating the flashing lights and vibration motor upon user interaction, and a pivotal stand on the rear surface of the housing, adapted to support the device at an angle for improved visibility of the alerts. The device can be wirelessly coupled to a remote computing device for transmitting detected glucose levels.

In this manner, the glucose monitoring device of the present invention accomplishes all of the forgoing objectives and provides a glucose monitoring device that designed specifically for deaf individuals. The device integrates advanced alert mechanisms, including bright flashing lights and continuous vibrations, to ensure that users with hearing impairments are effectively notified of abnormal glucose levels. This design not only enhances safety but also promotes independence and confidence in managing diabetes for deaf individuals.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a glucose monitor device for deaf individuals. The glucose monitoring device comprises a housing configured to house electronic components for monitoring glucose levels in a blood sample, a display screen on a front surface of the housing for displaying glucose level readings, a plurality of bright flashing lights disposed on the housing, wherein the lights are adapted to flash in bright colors to alert a user when the measured glucose level is abnormal, a vibration motor within the housing adapted to provide a tactile alert when the measured glucose level is abnormal, an acknowledgement button on the housing for deactivating the flashing lights and vibration motor upon user interaction, and a pivotal stand on the rear surface of the housing, adapted to support the device at an angle for improved visibility of the alerts.

In yet another embodiment, a glucose monitor device for deaf individuals is disclosed. The device includes a housing, a display screen on a front surface of the housing for displaying glucose level readings, a plurality (i.e., four) of bright flashing lights disposed on the housing, two lights on the top wall, one light on one side wall, and one light on the opposite side wall, wherein the lights are adapted to flash in a first color when the measured glucose level is less than a threshold range and in a second color when the measured glucose level is more than the threshold range, a vibration motor within the housing adapted to provide a tactile alert when the measured glucose level is outside the threshold range, an acknowledgement button on the housing for deactivating the flashing lights and vibration motor upon user interaction, and a pivotal stand on the rear surface of the housing, adapted to be positioned at different angles relative to the rear surface.

In another embodiment, the glucose monitoring device is adapted to wirelessly couple with a remote computing device. The glucose monitoring device is adapted to transmit real time glucose levels to the remote computing device.

In another aspect, a method for monitoring glucose levels and alerting deaf individuals is described. The method includes the steps of inserting a test strip into a blood sample test strip receiver of a glucose monitor device, measuring a glucose level in a blood sample using glucose monitoring circuitry within the device, comparing the measured glucose level with a predefined threshold range, generating a visual alert by activating a plurality of bright flashing lights on the housing of the device when the measured glucose level is outside the threshold range, generating a tactile alert by activating a vibration motor within the housing of the device when the measured glucose level is outside the threshold range, and deactivating the visual and tactile alerts upon user interaction with an acknowledgement button on the device.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of glucose monitor device of the present invention in accordance with the disclosed structure;

FIG. 2 illustrates another perspective view of the glucose monitoring device of the present invention in accordance with the disclosed structure;

FIG. 3 illustrates a block diagram view showing different electronic components included in the glucose monitoring device of the present invention in accordance with the disclosed structure;

FIG. 4 illustrates a perspective view showing the glucose monitoring device being placed bedside for easy visibility of alerts in accordance with the disclosed structure;

FIG. 5 illustrates a flow chart depicting a process of use of the glucose monitoring device for deaf of the present invention in accordance with the disclosed structure; and

FIG. 6 illustrates a perspective view showing the communication of the device with a remote computing device for transmitting recorded glucose measurements in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for an improved glucose monitoring device designed specifically for deaf individuals. There is also a long felt need in the art for a novel and inclusive glucose monitoring for deaf individuals by combining conventional functionality with enhanced alert mechanisms. Additionally, there is a long felt need in the art for a unique glucose monitoring device that includes bright flashing lights and continuous vibrations to provide effective notifications of abnormal glucose levels. Moreover, there is a long felt need in the art for a unique glucose monitoring system that facilitates monitoring of glucose by users, guardians, or medical practitioners. Finally, there is a long felt need in the art for a uniquely designed glucose monitoring device that enhances safety and promotes independence and confidence in managing diabetes for deaf individuals.

The present invention, in one exemplary embodiment, is a method for monitoring glucose levels and alerting deaf individuals. The method includes the steps of inserting a test strip into a blood sample test strip receiver of a glucose monitor device, measuring a glucose level in a blood sample using glucose monitoring circuitry within the device, comparing the measured glucose level with a predefined threshold range, generating a visual alert by activating a plurality of bright flashing lights on the housing of the device when the measured glucose level is outside the threshold range, generating a tactile alert by activating a vibration motor within the housing of the device when the measured glucose level is outside the threshold range, and deactivating the visual and tactile alerts upon user interaction with an acknowledgement button on the device.

Reference will now be made in detail to the present preferred embodiments of the invention, examples of which are illustrated in the accompanying drawings. Wherever possible, the same reference numerals are used in the drawings and the description to refer to the same or like parts.

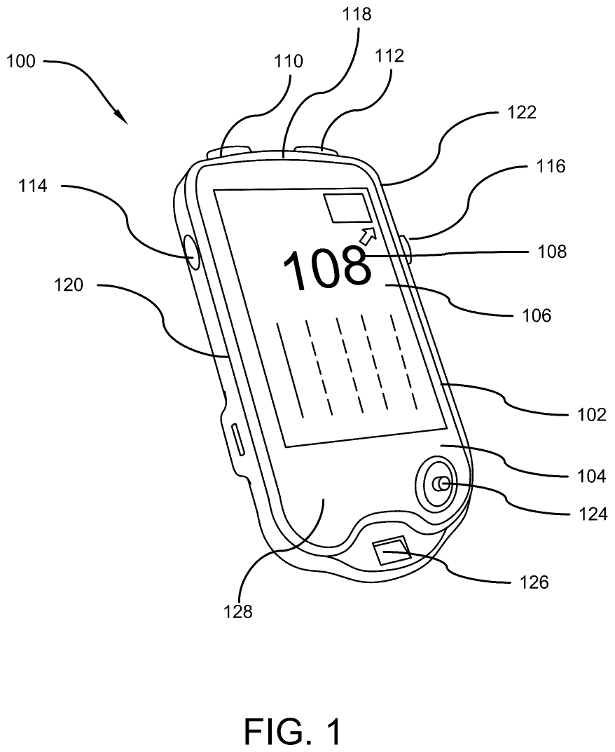

Referring initially to the drawings, FIG. 1 illustrates a perspective view of glucose monitor device of the present invention in accordance with the disclosed structure. The glucose monitoring device for deaf 100 of the present invention is designed as an improved glucose monitor device for effectively notifying deaf individuals. More specifically, the glucose monitoring device 100 includes a housing 102 which is adapted to house glucose monitoring circuitry for monitoring glucose level in a blood sample. The front surface 104 of the housing 102 includes a display screen 106 for displaying glucose level readings 108. The display screen 106 can also display other relevant information such as different levels of the glucose reading in a human body and can also be personalized based on age and gender of an individual.

The housing 102 includes a plurality of bright flashing lights 110, 112, 114, 116 disposed thereon. The lights 110-116 can be disposed at different appropriate positions on the housing 102 and preferably, two lights 110, 112 are disposed on the top wall 118 of the housing, a third light 114 is disposed on one side wall 120 and fourth light 116 is disposed on the opposite side wall 122 of the housing 102. The lights 110-116 are adapted to flash in bright colors to alert a user such as a deaf individual when the measured glucose level is abnormal.

In one embodiment, the flashing lights 110-116 are adapted to flash in a first color when the measured glucose level is less than a threshold range of glucose level and flash in a second color which is different from the first color when the measured glucose level is more than the threshold range. The lights 110-116 can flash in different styles including but not limited to continuous flashing, running flashing, and more.

A power button 124 is disposed on the housing 102 for activating and deactivating the device 100. A blood sample test strip receiver 126 is disposed on the front surface 104 for receiving and accommodating a test strip to monitor blood glucose level in the blood disposed on the test strip. The glucose monitoring device 100 is portable and lightweight and operates like a conventional device, measuring blood glucose levels.

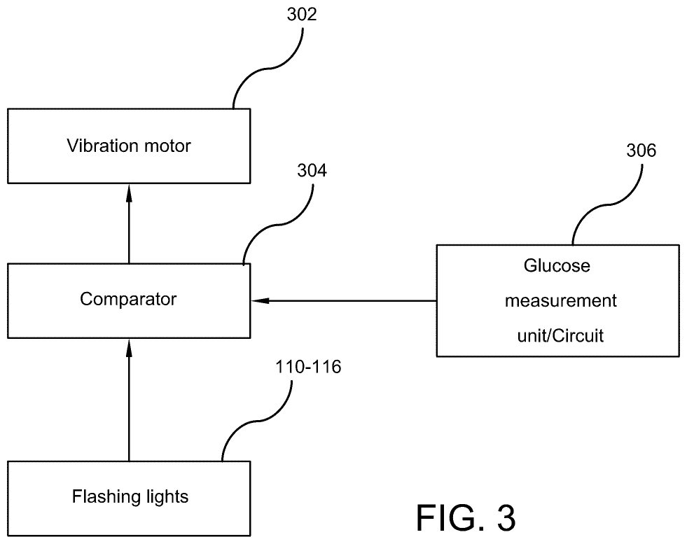

In addition to providing visual alert through the lights 110-116, the device 100 also provides a vibration (i.e., vibrator) to provide a tactile alert to a user as described in FIG. 3. It should be noted that in the present invention, both the lights and the vibration are designed to be persistent, continuing until the user acknowledges the alert by activating an acknowledgement button 128. In some embodiments, the user may acknowledge the alert by interacting with the touchscreen 106. When the button 128 is pressed, the lights 110-116 stop flashing and the device 100 stops vibrating, indicating that a user has noticed the alert to take the necessary steps to address their glucose levels.

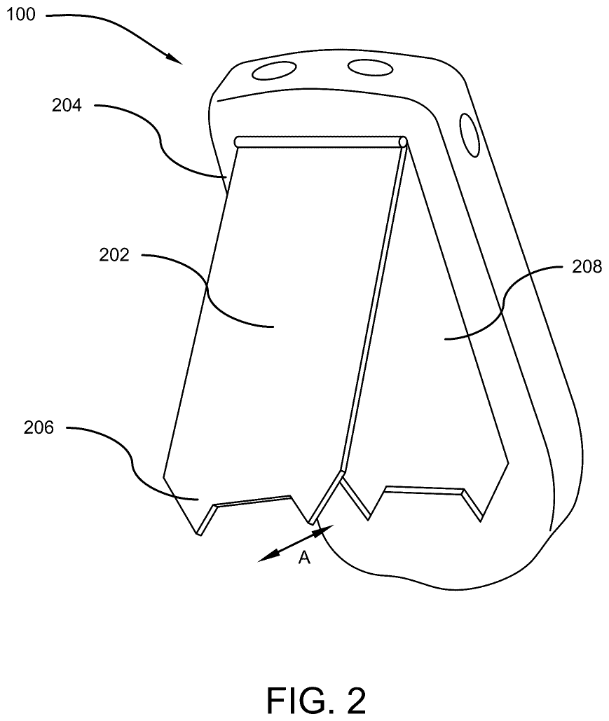

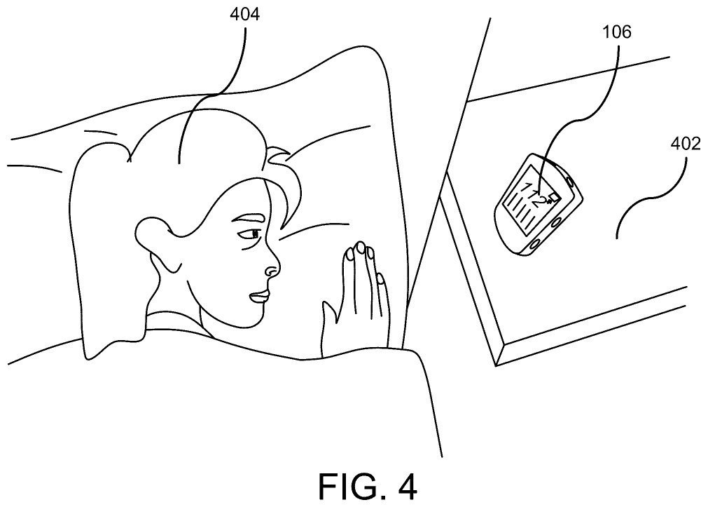

FIG. 2 illustrates another perspective view of the glucose monitoring device of the present invention in accordance with the disclosed structure. A pivotal stand 202 is disposed on the rear surface 204 of the housing 102 wherein the pivotal stand 202 is adapted to be at different angles relative to the rear surface 204 by moving the free end 206 of the stand 202 laterally as shown by the arrow A. The stand 202 is non-slippery and provides support to the device 100 at any appropriate angle as also illustrated in FIG. 4. The stand 202 enables a user to angle the device 100 towards their face, making it easier to see the flashing lights at night for receiving alerts. When the device 100 is to be stored such as in a cabinet, the stand 202 can be retracted to be accommodated in a slot 208 disposed in the rear surface 204 of the housing 102.

The device 100 can be made of any lightweight and durable material such as plastic or polycarbonate. Further, the display screen 106 can be made of LCD, OLED or any other similar technology. The device 100 can includes an integrated battery which can be replaceable and rechargeable. The device 100 can be configured for different glucose ranges and the threshold can be customized based on user preferences. The device 100 is ISO 15197 compliant and can have IPX4 rating.

FIG. 3 illustrates a block diagram view showing different electronic components included in the adaptive glucose monitor with visual and tactile alert mechanisms of the present invention in accordance with the disclosed structure. The device 100 includes a vibration motor 302 in the housing 102 for producing a consistent vibration in the device 100 when the glucose level is below or higher than the threshold glucose range. The vibration motor 302 is preferably a miniature electronic motor and consumes less power. The vibration helps in providing a tactile warning or alert to an individual.

The vibration motor 302 and the flashing lights 110-116 are coupled to a comparator 304 which is adapted to compare the glucose level measured from the glucose measurement unit 306 with the threshold range. The comparator 304 when detects that the measured glucose level is outside the range, then, an instruction signal is sent to the vibration motor 302 and the flashing lights 110-116 to activate both simultaneously. The acknowledgement button 128 when pressed by a user is adapted to close the circuit of the vibration motor 302 and the flashing lights 110-116 to deactivate both.

FIG. 4 illustrates a perspective view showing the glucose monitoring device being placed bedside for easy visibility of alerts in accordance with the disclosed structure. The stand 202 enables the device 100 to be placed at an angle such that the screen 106 makes an angle from about 110 degrees to about 130 degrees relative to the surface 402 on which the device 100 is positioned. The angled stand design ensures that the lights are visible even at night to a user 404, improving safety during sleep.

It should be noted that by incorporating multiple forms of alerts such as visual and vibration, the device 100 provides an inclusive solution that caters to the needs of deaf users without compromising on the functionality of a standard glucose monitor.

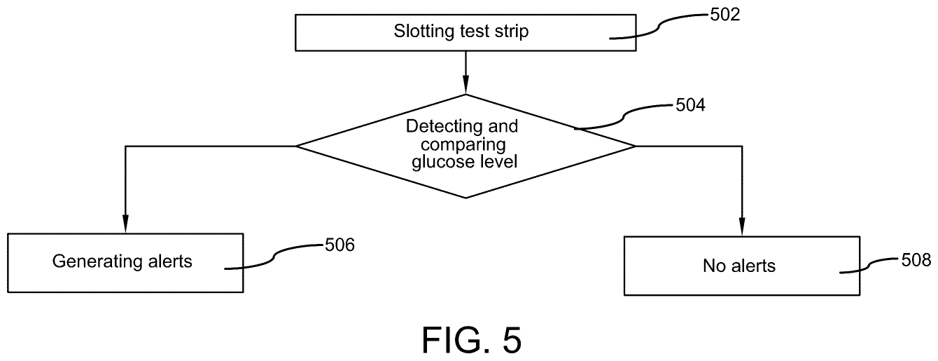

FIG. 5 illustrates a flow chart depicting a process of use of the adaptive glucose monitor with visual and tactile alert mechanisms of the present invention in accordance with the disclosed structure. Initially, a test strip is slotted in the blood sample test strip receiver 126 of the device 100 (Step 502). The device 100 then detects the glucose level and compares with the stored threshold range (Step 504). When the glucose level is detected to be outside the range, then the visual alert and vibrations are generated to alert a user (Step 506). When the glucose level is within the threshold range, then, the visual alert and vibrations are not generated (Step 508).

In some embodiment, the device 100 is configured to provide a reminder for measuring blood glucose by flashing lights in a customized flashing format. The reminder can be periodic at a specific frequency such as 12 hours, 24 hours, and more.

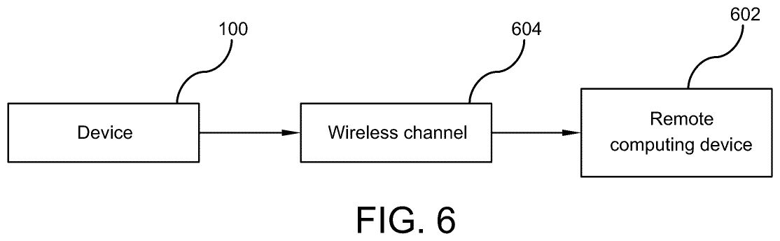

FIG. 6 illustrates a perspective view showing the communication of the device with a remote computing device for transmitting recorded glucose measurements in accordance with the disclosed structure. As illustrated, the device 100 can communicate with a remote computing device 602 using a wireless communication channel 604. The device 100 is adapted to provide real time glucose measurement to the remote computing device 602 wherein the remote computing device 602 can be of a user, a guardian, or a medical practitioner.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “adaptive glucose monitor with visual and tactile alert mechanisms”, “glucose monitor device”, “glucose monitor device for deaf”, and “device” are interchangeable and refer to the glucose monitoring device for deaf individuals 100 of the present invention.

Notwithstanding the forgoing the glucose monitoring device for deaf individuals 100 of the present invention can be of any suitable size and configuration as is known in the art without affecting the overall concept of the invention, provided that it accomplishes the above stated objectives. One of ordinary skill in the art will appreciate that the glucose monitoring device for deaf individuals 100 as shown in the FIGS. are for illustrative purposes only, and that many other sizes and shapes of the glucose monitoring device for deaf individuals 100 are well within the scope of the present disclosure. Although the dimensions of the glucose monitoring device for deaf individuals 100 are important design parameters for user convenience, the glucose monitoring device for deaf individuals 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A glucose monitoring device comprising;
a housing having a front surface including a display screen;
a blood sample test strip receiver;
a vibrator;
a glucose monitor; and
a plurality of lights;
wherein said blood sample test strip receiver accommodates a test strip to monitor a blood glucose level in a blood sample disposed on a test strip;
wherein said housing houses said glucose monitor;
wherein said glucose monitor having circuitry for monitoring the blood glucose level in the blood sample;
wherein said display screen for displaying the blood glucose level from said glucose monitor;
wherein said housing having said plurality of lights disposed on an outer surface of said housing;
wherein said plurality of lights having actuated flashing lights when the blood glucose level is outside of a threshold glucose range; and
further wherein said vibrator providing a vibration to said housing to provide a tactile alert to a user holding the glucose monitoring device when the blood glucose level is outside of said threshold glucose range.

2. The glucose monitoring device of claim 1, wherein a pair of lights disposed on a top wall of said housing.

3. The glucose monitoring device of claim 2, wherein a third light disposed on a first side wall.

4. The glucose monitoring device of claim 3, wherein a fourth light disposed on an opposed second side wall.

5. The glucose monitoring device of claim 1, wherein said flashing lights flash in a first color when the glucose level reading is less than said threshold glucose range.

6. The glucose monitoring device of claim 5, wherein said flashing lights flash in a second color when glucose level reading is more than said threshold glucose range.

7. The glucose monitoring device of claim 6, wherein said first color is different than said second color.

8. A glucose monitoring device comprising;
a housing having a front surface including a display screen, and a back surface;
a blood sample test strip receiver;
a vibrator;
a glucose monitor;
a plurality of lights; and
a pivotal stand;
wherein said blood sample test strip receiver accommodates a test strip to monitor a blood glucose level in a blood sample disposed on a test strip;
wherein said housing houses said glucose monitor;
wherein said glucose monitor having circuitry for monitoring the blood glucose level in the blood sample;
wherein said display screen for displaying the blood glucose level from said glucose monitor;
wherein said housing having said plurality of lights disposed on an outer surface of said housing;
wherein said plurality of lights having actuated flashing lights when the blood glucose level is outside of a threshold glucose range;
wherein said vibrator providing a vibration to said housing to provide a tactile alert to a user holding the glucose monitoring device when the blood glucose level is outside of said threshold glucose range;
wherein said pivotal stand disposed on said back surface of said housing; and
further wherein said pivotal stand adaptable at different angles relative to said back surface.

9. The glucose monitoring device of claim 8, wherein a pair of lights disposed on a top wall of said housing.

10. The glucose monitoring device of claim 9, wherein a third light disposed on a first side wall.

11. The glucose monitoring device of claim 10, wherein a fourth light disposed on an opposed second side wall.

12. The glucose monitoring device of claim 8, wherein said flashing lights flash in a first color when the glucose level reading is less than said threshold glucose range.

13. The glucose monitoring device of claim 12, wherein said flashing lights flash in a second color when glucose level reading is more than said threshold glucose range.

14. The glucose monitoring device of claim 13, wherein said first color is different than said second color.

15. A glucose monitoring device comprising;
a housing having a front surface including a display screen;
a blood sample test strip receiver;
a vibrator;
a glucose monitor; and
a plurality of lights;
wherein said blood sample test strip receiver accommodates a test strip to monitor a blood glucose level in a blood sample disposed on a test strip;
wherein said housing houses said glucose monitor;
wherein said glucose monitor having circuitry for monitoring the blood glucose level in the blood sample;
wherein said display screen for displaying the blood glucose level from said glucose monitor;
wherein said housing having said plurality of lights disposed on an outer surface of said housing;
wherein said plurality of lights having actuated flashing lights when the blood glucose level is outside of said threshold glucose range;
wherein said vibrator providing a vibration to said housing to provide a tactile alert to a user holding the glucose monitoring device when the blood glucose level is outside of said threshold glucose range; and
further wherein said threshold glucose range is a selectable range by a user of the glucose monitoring device.

16. The glucose monitoring device of claim 15, wherein a pair of lights disposed on a top wall of said housing, and further wherein a third light disposed on a first side wall.

17. The glucose monitoring device of claim 16, wherein a fourth light disposed on an opposed second side wall.

18. The glucose monitoring device of claim 15, wherein said flashing lights flash in a first color when the glucose level reading is less than said threshold glucose range.

19. The glucose monitoring device of claim 18, wherein said flashing lights flash in a second color when glucose level reading is more than said threshold glucose range.

20. The glucose monitoring device of claim 19, wherein said first color is different than said second color.
